(19) United States
(12) Plant Patent Application Publication
(10) Pub. No.: US 2021/0022280 P1
(43) Pub. Date: Jan. 21, 2021

(54) Cannabis plant honey hiker

(50) ;

(71) Applicant: MJ Brain Bank, LLC, Denver, CO (US)

(72) Inventor: Kevin Sullivan, Broomfield, CO (US)

(21) Appl. No.: 16/501,982

(22) Filed: Jul. 15, 2019

(51) Int. Cl. A01H 6/28

(52) U.S. Cl. PLT/258

(57) ABSTRACT

‘Honey Hiker’ is new Cannabis plant variety that has a combination of characteristics that include high potency, sweet aroma, greater tolerance for fungal diseases, and more vigor as compared to the parents, the closest varieties.

Genus and species: The Cannabis variety of this invention is botanically identified as Cannabis sativa.

Variety denomination: The variety denomination is ‘Honey Hiker’.

### BACKGROUND OF THE INVENTION

The invention refers to a new plant variety of Cannabis (Cannabis sativa) named ‘Honey Hiker’. The new Cannabis variety is product of a planned and controlled breeding program for new Cannabis varieties. The objective of the breeding program is to select new Cannabis varieties with a combination of desired vigor, potency, aroma, bud structure/form, yield, and tolerance or resistance to diseases.

The new Cannabis variety is a product of a planned breeding program intended to combine some of the desirable characteristics of two existing varieties: Retro Boogie (Cannabis indica) and Long Bottom Leaf (Cannabis sativa). The variety arose from a cross between the parental varieties; using Long Bottom Leaf as the pollen donor. The parental cross was made in April 2017. Single plants were selected and reproduced by cuttings using traditional horticultural plugs, trays and domes. One of the reproduced plants showed pronounced vigor and was selected as and named ‘Honey Hiker’. This plant was transferred to the production on July 2017 and has been asexually reproduced by cuttings since that time. The distinctive characteristics of the variety are stable through successive generations.

For asexual reproduction, axillary branches of the plant were excised from the mother plant. The cuttings were treated with indole 3 butyric acid; the cuttings were allowed to root over a period of three weeks at a temperature of 77 degrees F., relative humidity of 60%-80% and constant illumination at an intensity of 100 PPFD.

### BRIEF SUMMARY OF THE INVENTION

The following traits have been repeatedly observed and are determined to be unique characteristics of ‘Honey Hiker’ which in combination distinguish this Cannabis plant as a new and distinct variety, as compared to the parents, the closet varieties:
1. The new variety has higher potency as compared to the parents, the closest varieties.
2. The new variety has a sweet aroma as compared to the earthy or spicy aroma in the parents.
3. The new variety has a greater tolerance for fungal diseases such as powdery mildew.
4. The new variety has more vigor as compared to the parents, the closest varieties.

The traits of the new variety was tested at 2600 West Barberry Pl. Denver, Colo., U.S.A.

### BRIEF DESCRIPTION OF THE PHOTOGRAPHS

The accompanying photographs illustrate the appearance of the new Cannabis variety when the plants were at 63 days after flower initiation. The colors in the photographs are depicted as nearly true as is reasonably possible to obtain in colored reproductions of this type. Colors as shown in the photographs may differ slightly from the color values cited in the detailed botanical description.

FIG. 1 shows a branch of a plant of the new variety.

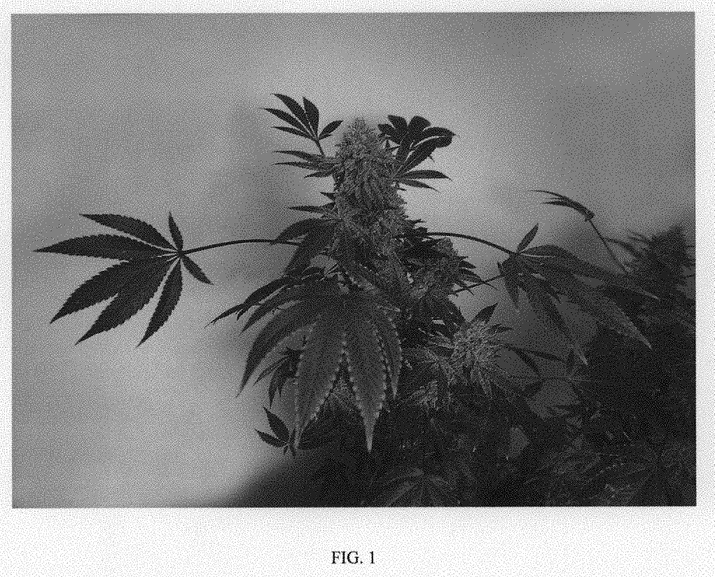

FIG. 2 shows a single plant of the new variety.

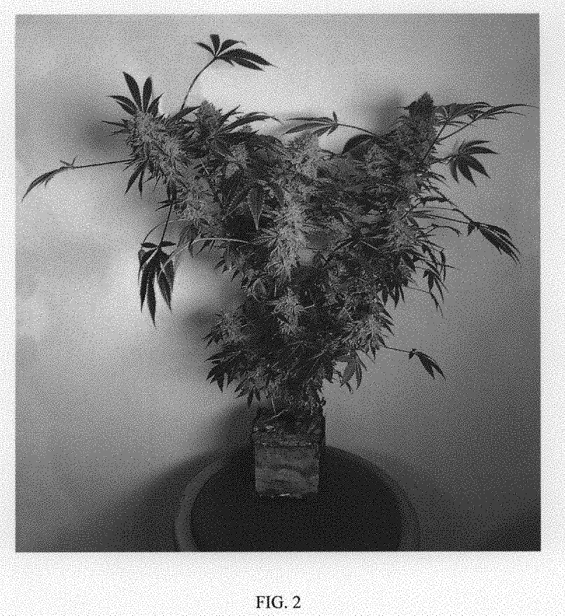

FIG. 3 shows flowers of a plant of the new variety.

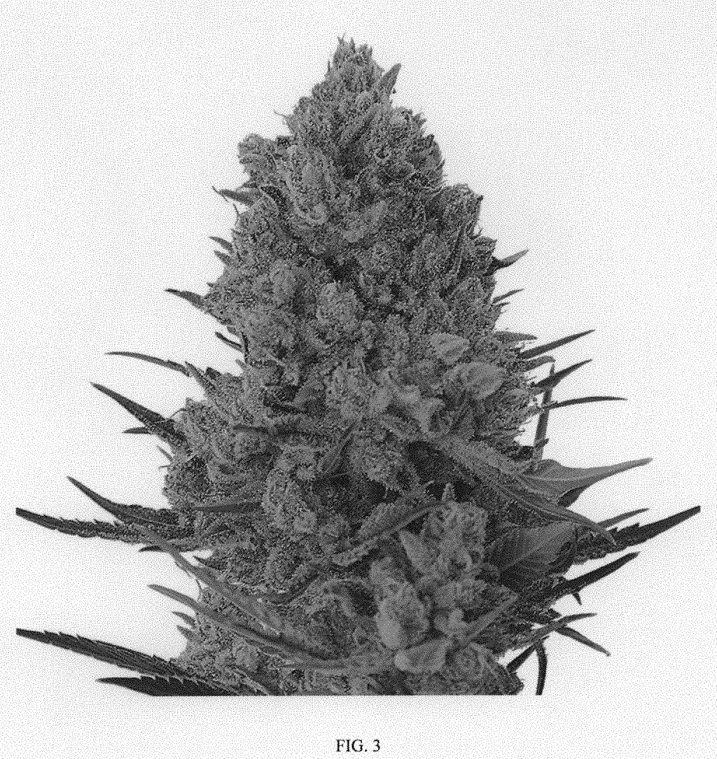

FIG. 4 shows a dried bud of a plant of the new variety.

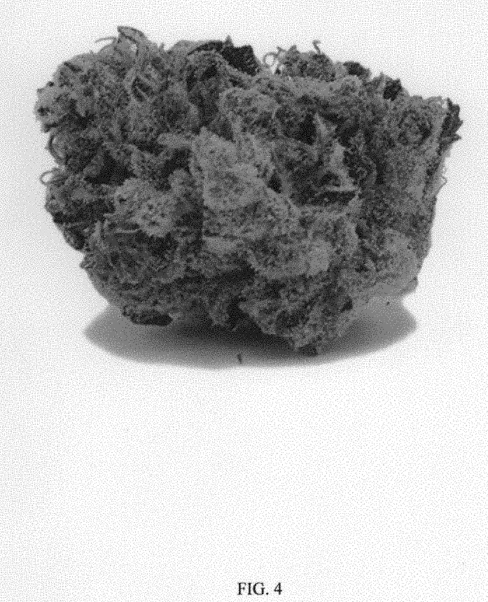

### DETAILED BOTANICAL DESCRIPTION

The following description is based on observations made in February of 2016 on plants produced according to an ordinary growth cycle. However, the new Cannabis variety ‘Honey Hiker’ has not been observed under all possible environmental conditions. The phenotype of the new variety may vary with variations in environmental conditions such as temperature, light intensity and day length without any change in the genotype of the plant.

The photographs and description are taken from plants grown at the 2600 West Barberry Pl. in Denver, Colo., U.S.A. Following rooting, the cuttings were transplanted into Hydroton, and were grown for approximately 6 weeks or until roots emerged. The plants were then allowed to grow additional 9 weeks. During growth, all plants were fed and watered with nutrients from the Botanicare Kind line. Carbon dioxide concentration was measured and supplemented as required, between 900-1500 ppm. Vegetative growth was at a temperature of 78, and relative humidity of 45 with constant illumination of 600 PPFD provided by 600 W Metal Halide grow lights. After adequate vegetative growth had been attained, flowering was induced by changing the photoperiod to 12 hours of light and 12 hours of darkness

Plants were observed on day 63 after the flower induction and the botanical description thereof is provided below. Characteristics described represent those which are typical for the new variety. Measurements and numerical values represent averages for 12 typical plants and plant parts. The actual measurements of any individual plant of plant parts, or any group of plants of plant parts, may vary from the stated averages.
Plant description:
I. General:

| | | Parental | | Check Variety |
|---|---|---|---|---|
| | | variety | Parental variety | (Cannabis |
| Characteristic | New Variety | (Female plant) | (Pollen donor) | Sativa) |
| Plant life forms | Herbaceous | Herbaceous | Herbaceous Plant | Herbaceous |
| | Plant | Plant | | Plant |
| Plant growth habit | Upright, tap- | Upright, tap- | Upright, tap- | Upright, tap- |
| | rooted annual | rooted annual | rooted annual | rooted annual |
| | plant | plant | plant | plant |
| Plant origin | F1 hybrid | F1 Cannabis | F1 hybrid between | F1 hybrid |
| | between two | Subspecies (G- | two Cannabis | between two |
| | Cannabis | 13 × | Subspecies (SFV | Cannabis |
| | Subspecies | Hashplant) | OG Kush × Jack's | Subspecies |
| | ((G-13 × | | Cleaner 2) | (Lambs Bread × |
| | Hashplant) × | | | Sour Diesel) |
| | (SFV OG | | | |
| | Kush × Jack's | | | |
| | Cleaner 2) | | | |
| Plant propagation | Asexual | Asexual | Asexual | Asexual |
| Propagation ease | Easy¹ | Easy | Moderate² | Moderate |
| Height | Variable; 1 | Variable; 1 | Variable; 1 meter | Variable; 1 |
| (Unit: m or cm) | meter | meter | | meter |
| Width | Variable; | Variable; 0.75 | Variable; 0.75 | Variable; 0.75 |
| (Unit: m or cm) | 0.75 meters | meters | meters | meters |
| Plant vigor | High | High | Medium | High |
| Time to Harvest | 18-20 weeks | 18-20 weeks | 18-20 weeks | 18-20 weeks |
| (Seed to Harvest) | | | | |
| Resistance to pests or | No | No | No | No |
| diseases? | | | | |
| Is this plant a | No | No | No | No |
| Genetically Modified | | | | |
| Organism? | | | | |

¹Easy refers to plants that don't require any special products, chemicals or hormones for rooting. Roots will also show up within 10-14 days.
²Moderate refers to plants that may take 14-21 days with an increase in rooting after additional hormone or nutrient has been introduced.

II. Leaf/foliage:

| | | Parental | | |
|---|---|---|---|---|
| | | variety | | |
| | | (Female | Parental variety | |
| Characteristic | New Variety | plant) | (Pollen donor) | Check Variety |
| Leaf arrangement | Alternate | Alternate | Alternate | Alternate |
| (Phyllotaxy) | | | | |
| Leaf shape | Palmately | Palmately | Palmately | Palmately |
| | compound | compound | compound | compound |
| Leaf structure | Palmately | Palmately | Palmately | Palmately |
| | compound | compound | compound | compound |
| Leaf margins | Coarsely | Coarsely | Coarsely serrated | Coarsely |
| | serrated | serrated | pointing towards | serrated |
| | pointing | pointing | tip of leaflet | pointing |
| | towards tip of | towards tip | | towards tip of |
| | leaflet | of leaflet | | leaflet |
| Leaf hairs | Absence | Absence | Absence | Absence |
| (Presence or absence) | | | | |
| Leaf length with | 20 | 16 | 16 | 18 |
| petiole at maturity | | | | |
| (Unit: cm) | | | | |
| Petiole length at | 12 | 10 | 11 | 12 |
| maturity | | | | |
| (Unit: cm) | | | | |
| Stipule length at | 1 | 1 | 1 | 1 |
| maturity | | | | |
| (Unit: cm) | | | | |
| Stipule shape | Long, bulbous | Long, | Long, bulbous | Long, bulbous |
| | bases with | bulbous | bases with | bases with |
| | acuminate | bases with | acuminate | acuminate |
| | (tapering | acuminate | (tapering concave | (tapering |
| | concave to | (tapering | to apex) shape | concave to |
| | apex) shape | concave to | | apex) shape |
| | | apex) shape | | |
| Number of leaflets | 5-9 | 5-9 | 5-9 | 5-9 |
| Middle largest | 8 | 6 | 8 | 8 |
| (longest) leaflet length | | | | |
| (Unit: cm) | | | | |
| Middle largest | 2 | 2 | 2 | 2 |
| (longest) leaflet width | | | | |
| (Unit: cm) | | | | |
| Middle largest | 4:1 | 3:1 | 4:1 | 4:1 |
| (longest) leaflet | | | | |
| length/width ratio | | | | |
| Number teeth of | 12 | 13 | 11 | 13 |
| middle leaflet | | | | |
| (average) | | | | |
| Leaf (upper side) color | 141C | 141C | 141C | 141C |
| (RHS³ No.) | | | | |
| Leaf (lower side) color | 143A | 143A | 143A | 143A |
| (RHS No.) | | | | |
| Leaf glossiness | Medium | Medium | Medium | Medium |
| Vein/midrib shape | Even, not | Even, not | Even, not | Even, not |
| (general description) | alternate. | alternate. | alternate. Slightly | alternate. |
| | Slightly points | Slightly | points towards tip | Slightly points |
| | towards tip just | points | just like the | towards tip just |
| | like the | towards tip | serrations | like the |
| | serrations | just like the | | serrations |
| | | serrations | | |
| Vein/midrib color | Light green | Light green | Light green | Light green |
| | (RHS 140B) | (RHS 140B) | (RHS 140B) | (RHS 140B) |
| Aroma | Sweet | Earthy | Herbal/Spicy | Gas/Fuel, |
| (general description) | | | | Earthy |

3RHS stands for Royal Horticultural Colour Chart, which can be found at: http://rhscforgfree.com

III. Stem:

| | | Parental | | |
|---|---|---|---|---|
| | | variety | Parental | |
| | | (Female | variety | |
| Characteristic | New Variety | plant) | (Pollen donor) | Check Variety |
| Stem shape | hollow, large, | hollow, | hollow, large, | hollow, large, |
| (general description) | rugose, | large, rugose, | rugose, | rugose, punctuate, |
| | punctuate, | punctuate, | punctuate, | minutely |
| | minutely | minutely | minutely | glandular, ribbed |
| | glandular, ribbed | glandular, | glandular, | with ribs running |
| | with ribs running | ribbed with | ribbed with | parallel to stem |
| | parallel to stem | ribs running | ribs running | |
| | | parallel to | parallel to stem | |
| | | stem | | |
| Stem diameter at | 1.25 | 1 | 1.5 | 1 |
| base | | | | |
| (Unit: cm) | | | | |
| Stem color | 144D | 144D | 144D | 144D |
| (RHS No.) | | | | |

IV. Inflorescence (female/pistillate flowers):

| | | Parental | Parental | |
|---|---|---|---|---|
| | New Variety | variety (Female | variety | |
| Characteristic | (Female plant) | plant) | (Pollen donor) | Check Variety |
| Flowering (blooming) | Dioecious. | Dioecious. | Dioecious. | Dioecious. |
| habit (General | Forms a tall | Forms a tall | Small | |
| description) | interconnected | interconnected | inflorescences, | |
| | cola or terminal | cola or terminal | tightly packed | |
| | bud | bud | against stem and | |
| | | | separated all | |
| | | | along plants | |
| | | | branches. | |
| Proportion of female | 50% | N/A | N/A | N/A |
| plants | | | | |
| (50% or less?) | | | | |
| Inflorescence | Above | Above | Above | Above |
| position | | | | |
| Flower arrangement | Overlapping | Overlapping | Overlapping | Overlapping |
| Number of flowers | 240 | 190 | 210 | 200 |
| per plant | | | | |
| Flower shape | sessile and are | sessile and are | sessile and are | sessile and are |
| | borne in | borne in | borne in | borne in |
| | racemes (an | racemes (an | racemes (an | racemes (an |
| | indeterminate, | indeterminate, | indeterminate, | indeterminate, |
| | branched type of | branched type of | branched type of | branched type of |
| | inflorescence | inflorescence | inflorescence | inflorescence |
| | bearing flowers | bearing flowers | bearing flowers | bearing flowers |
| | with short floral | with short floral | with short floral | with short floral |
| | stalks called | stalks called | stalks called | stalks called |
| | pedicels along | pedicels along | pedicels along | pedicels along |
| | the axis). Each | the axis). Each | the axis). Each | the axis). Each |
| | flower has a | flower has a | flower has a | flower has a |
| | small green | small green | small green | small green |
| | bract enclosing | bract enclosing | bract enclosing | bract enclosing |
| | the ovary with | the ovary with | the ovary with | the ovary with |
| | two long, | two long, | two long, | two long, |
| | slender stigmas | slender stigmas | slender stigmas | slender stigmas |
| | projecting well | projecting well | projecting well | projecting well |
| | above the bract. | above the bract. | above the bract. | above the bract. |
| | And the flower | And the flower | And the flower | And the flower |
| | looks urceolate | looks urceolate | looks urceolate | looks urceolate |
| | (urn-shaped). | (urn-shaped). | (urn-shaped). | (urn-shaped). |
| Flower (individual | 3 | 3 | 3 | 3 |
| pistillate) length | | | | |
| (Unit: mm) | | | | |
| Flower (compound | 2 | 2 | 1.5 | 2 |
| cyme) diameter | | | | |
| (Unit: cm) | | | | |
| Corolla | Round | Round | Round | Round |
| (general description) | | | | |
| Corolla Color | 142C | 142C | 142C | 142C |
| (RHS No.) | | | | |
| Bract shape | Round at the | Round at the | Round at the | Round at the |
| (general description) | base but tapered | base but tapered | base but tapered | base but tapered |
| | towards the tip | towards the tip | towards the tip | towards the tip |
| Bract color | 142C | 142C | 142C | 142C |
| (RHS No.) | | | | |
| Calyx shape | Round | Round | Round | Round |
| (general description) | | | | |
| Calyx color | 142C | 142C | 142C | 142C |
| (RHS No.) | | | | |
| Stigma shape | Curved and | Curved and | Curved and | Curved and |
| (general description) | wavy, thin | wavy, thin | wavy, thin | wavy, thin |
| Stigma length | 0.6 | 0.6 | 0.6 | 0.6 |
| (Unit: cm) | | | | |
| Stigma color | closely | closely | closely | closely |
| (RHS No.) | resembling an | resembling an | resembling an | resembling an |
| | off white or | off white or | off white or | off white or |
| | NN155D | NN155D | NN155D | NN155D |
| Trichome shape | Glandular, thus | Glandular, thus | Glandular, thus | Glandular, thus |
| (general description) | it has a stalk that | it has a stalk that | it has a stalk that | it has a stalk that |
| | supports | supports | supports | supports |
| | secretory head | secretory head | secretory head | secretory head |
| Trichome color (RHS | Mature cap = | Mature cap = | Mature cap = | Mature cap = |
| No.) | N172C | N172C | N172C | N172C |
| | Stalk = | Stalk = | Stalk = | Stalk = |
| | Clear/Translucent | Clear/Translucent | Clear/Translucent | Clear/Translucent |
| | closely | closely | closely | closely |
| | resembling an | resembling an | resembling an | resembling an |
| | off white or | off white or | off white or | off white or |
| | NN155D | NN155D | NN155D | NN155D |
| Terminal bud shape | Rounded top | Large | Small, rounded | Round but with |
| (general description) | buds with | interconnected | and slightly flat | textured |
| | slightly fox | cola or terminal | on top. | projections (fox |
| | tailing | bud. Conical | | tails). Spikey |
| Terminal bud | 142C | 142C | 142C | 142C |
| color (RHS No.) | | | | |
| Pedicel | absence | absence | absence | absence |
| (Presence or absence) | | | | |
| Staminate shape | N/A | N/A | Round sacks of | N/A |
| | | | modular | |
| | | | composition like | |
| | | | the hand of a | |
| | | | banana but | |
| | | | originating from | |
| | | | a set of stems | |
| | | | similar to grapes | |
| Pollen description | N/A | N/A | Typical yellow | N/A |
| | | | pollen with | |
| | | | musty smell | |
| Seed description | Starts off white | Starts off white | N/A | Starts off white |
| | and turns to | and turns to | | and turns to |
| | Brown with | Brown with | | Brown with |
| | black or dark | black or dark | | black or dark |
| | brown stripes | brown stripes | | brown stripes |
| Petal description | absence | absence | absence | absence |
| Tetrahydro- | 25-28% THC | 22-24% THC | N/A (male) | 20-24% THC |
| cannabinolic | | | | |
| acid (THC) content | | | | |

V. Other characteristics and culture:

| | | Parental | | |
|---|---|---|---|---|
| | | variety | Parental | |
| | New Variety | (Female | variety | |
| Characteristic | (Female plant) | plant) | (Pollen donor) | Check Variety |
| Time period and | Blooms in 9-11 | Blooms in 9- | Blooms in 9-11 | Blooms in 9-11 |
| condition of | weeks after | 11 weeks | weeks after | weeks after |
| flowering/blooming | cutting-and- | after cutting- | cutting-and- | cutting-and- |
| | rooting when | and-rooting | rooting when | rooting when |
| | more than 12 | when more | more than 12 | more than 12 |
| | hours light is | than 12 | hours light is | hours light is |
| | applied to induce | hours light is | applied to | applied to induce |
| | flowering | applied to | induce | flowering |
| | | induce | flowering | |
| | | flowering | | |
| Hardiness of plant | High | High | Medium | Medium |
| Breaking action⁴ | High | High | Medium | Medium |
| Rooting rate after | 9 days | 9 days | 9 days | 9 days |
| cutting/cloning | | | | |
| (under certain or | | | | |
| specific condition) | | | | |

⁴refers to how strong the stem is against the breakage such as fibrous, strong, flexible, resistant to breakage, etc.

What is claimed is:

1. A new and distinct variety of Cannabis plant having the characteristics substantially as described and illustrated herein.
